(19) United States
(12) Patent Application Publication
(10) Pub. No.: US 2023/0104030 A1
(43) Pub. Date: Apr. 6, 2023

(54) DETECTION OF SLOWING PATTERNS IN EEG DATA

(71) Applicant: Nanyang Technological University, Singapore (SG)

(72) Inventors: Justin DAUWELS, Singapore (SG); Wei Yan PEH, Singapore (SG); John THOMAS, Singapore (SG); Michel BIRNBAUM, Singapore (SG)

(21) Appl. No.: 17/905,994

(22) PCT Filed: Mar. 4, 2021
(86) PCT No.: PCT/SG2021/050111

§ 371 Date: Sep. 9, 2022

(30) Foreign Application Priority Data
Mar. 9, 2020 (SG) 10202002129U

(51) Int. Cl. A61B 5/372; A61B 5/00; A61B 5/384; A61B 5/291

(52) U.S. Cl. CPC A61B 5/372; A61B 5/7214; A61B 5/7267; A61B 5/7246; A61B 5/384; A61B 5/291; A61B 5/7275

(57) ABSTRACT

A method for detecting the presence of slowing patterns in an EEG sample comprising a plurality of channels of EEG signals, each channel comprising one or more segments, the method comprising: obtaining a first classifier that is trained to classify EEG samples as containing abnormal slow waves or not; performing a sequence of artifact removal processes on the EEG sample to generate a preprocessed EEG sample; extracting a first feature set from the preprocessed EEG sample; and passing the first feature set to the first classifier to predict whether the EEG sample contains abnormal slow waves or not; wherein the sequence of artifact removal processes comprises removal of one or more ocular artifacts and removal of one or more electrode artifacts.

### CROSS REFERENCE

The present application is a 371 national stage filing of International PCT Application No. PCT/SG2021/050111 by DAUWELS et al. entitled “DETECTION OF SLOWING PATTERNS IN EEG DATA,” filed Mar. 4, 2021, which is assigned to the assignee hereof, and which is expressly incorporated by reference in its entirety herein.

### TECHNICAL FIELD

The present disclosure relates to detection of slowing patterns in EEG data, for example, for diagnosis of an underlying neurological condition, or monitoring brain activity during anesthesia.

### BACKGROUND

An electroencephalogram (EEG) is a recording of the electrical activity of the brain collected by placing electrodes on the scalp of a subject. Abnormal patterns in EEG recordings can be indicative of an underlying neurological problem. One important class of abnormal patterns is slowing patterns. “Slowing”, in the context of an EEG waveform, means a decrease in frequency in part of the waveform. The presence of slowing in EEG data may imply different possible cerebral dysfunction forms such as brain lesion, epilepsy, stroke, Alzheimer's, autism, and brain haemorrhage.

EEG slowing often has important implications for the location of Central Nervous System (CNS) abnormalities and/or the prognosis for neurological recovery. Slowing can be specific (focal slowing) or unspecific (generalized slowing). The level of clinical cerebral disturbance is correlated with the severity and duration of slowing. During EEG recordings, slowing usually appears in the delta (1-4 Hz) and theta (4-8 Hz) frequency bands, with delta slowing being more severe than theta slowing. Additionally, slowing can last for different durations. An EEG may exhibit a rare short burst of slowing, intermittent and occasional slowing, or continuous, frequent, and persistent slowing.

Slowing can also occur in the higher frequency bands, such as the alpha band, within a comatose patient. The severity of slowing also depends on the patient's age. For example, it is typical for older patients to present slower EEGs.

In current clinical practice, slow waves must be visually identified by neurologists. This process is tedious and time-consuming. Moreover, there is no clear consensus among experts of what constitutes slowing in EEG. Slowing detection can be difficult as the slow waves exhibit a large morphological variety across patients.

In view of these difficulties, there exists a great need for automated slowing detection in EEG to classify the existence and degree of slowing. Most previous research has been focused on slow sleep waves (SWS). However, analysis of SWS cannot assist with neurological prognosis or diagnosis, such as in relation to stroke or brain trauma. Several methods have indirectly used slowing to develop a neurological diagnosis, but none of these have been validated on a sizable dataset.

### SUMMARY

The present invention relates to a method for detecting the presence of slowing patterns in an EEG sample comprising a plurality of channels of EEG signals, each channel comprising one or more segments, the method comprising:

obtaining a first classifier that is trained to classify EEG samples as containing abnormal slow waves or not;

performing a sequence of artifact removal processes on the EEG sample to generate a preprocessed EEG sample;

extracting a first feature set from the preprocessed EEG sample; and

passing the first feature set to the first classifier to predict whether the EEG sample contains abnormal slow waves or not;

wherein the sequence of artifact removal processes comprises removal of one or more ocular artifacts and removal of one or more electrode artifacts.

By applying a sequence of artifact removal processes, different types and sources of artifact can be removed from the EEG signal, thereby improving the accuracy of slowing detection.

In some embodiments, removal of one or more electrode artifacts comprises: identifying and removing low signal segments; identifying and removing disconnected segments; and/or identifying and removing abnormal high-amplitude segments.

Removal of one or more ocular artifacts may comprise removal of eye blink artifacts. For example, removal of eye blink artifacts may comprise determining a correlation between an Fp1 channel of the plurality of channels and an Fp2 channel of the plurality of channels in the preprocessed EEG sample in respective segments of said one or more segments; and removing, from the preprocessed EEG sample, any segments for which the correlation exceeds a threshold.

Eye blink artifacts may cause false positives when performing slowing detection. Accordingly, by removing such artifacts, the accuracy of detection is improved, and significantly improving the interpretability of the EEG.

In some embodiments, the first classifier is applied separately to each of said channels to obtain a plurality of channel-wise slowing predictions.

By obtaining a plurality of channel-wise slowing predictions, for example for each segment of each channel, it is possible to determine the slowing percentage in each channel. This allows for generation of EEG scalp plots of the slowing distribution and percentage across the scalp, which aids in visualization of the localization of slowing in an EEG.

The method may comprise obtaining a second classifier that is trained to classify the one or more segments as containing abnormal slow waves based on a second feature set that is extracted from the first feature set and/or from the plurality of channel-wise slowing predictions; and passing the second feature set to the second classifier to obtain a slowing prediction for the one or more segments or for the EEG sample as a whole.

In some embodiments, the first feature set comprises one or more spectral features, wherein each spectral feature is based on at least one relative power value that is a ratio of a power in a frequency band to a total power in one of the channels.

The one or more spectral features may comprise one or more of the following power ratios: power ratio index, PRI=(δ+θ)/(α+β); delta alpha ratio, DAR=δ/α; theta alpha ratio, TAR=θ/α; and theta beta ratio, TBAR=θ/(α+β); where α is relative power in the α frequency band, β is relative power in the β frequency band, δ is relative power in the δ frequency band, and θ is relative power in the θ frequency band.

In some embodiments, the second feature set comprises one or more statistical properties of the plurality of channel-wise predictions.

In some embodiments, the second feature set comprises one or more statistical properties of the one or more relative power values and/or the one or more power ratios.

The statistical properties may comprise one or more of: a histogram; a mean; a standard deviation; a minimum; a maximum; a range; a standard deviation of the gradient; and/or a standard deviation of the curvature.

In some embodiments, the first classifier is a support vector machine, a binary classifier based on thresholding, or logistic regression.

In other embodiments, the first classifier is a convolutional neural network (CNN).

In some embodiments, the second classifier is a support vector machine, logistic regression, or random forests.

The present invention also relates to a system for detecting the presence of slowing patterns in EEG data, the system comprising:

memory; and

at least one processor in communication with the memory;

wherein the memory has stored thereon computer-readable instructions for causing the at least one processor to perform a method as disclosed herein.

The present invention further relates to non-transitory computer-readable storage having stored thereon instructions for causing at least one processor to perform a method as disclosed herein.

### BRIEF DESCRIPTION OF THE DRAWINGS

Some embodiments of a system and method for detection of slowing patterns in EEG data, in accordance with present teachings will now be described, by way of non-limiting example only, with reference to the accompanying drawings in which:

FIG. 1 is a block diagram of an example system for detection of slowing patterns in EEG data;

FIG. 2 is a schematic depiction of placement of EEG electrodes in the international 10-20 system;

FIG. 3 is a flow diagram of an example method for training a classifier for detection of slowing patterns in EEG data;

FIG. 4A is a flow diagram of an example preprocessing method;

FIG. 4B is a flow diagram of an example artifact removal process;

FIG. 5 is a flow diagram of an example method for detection of slowing patterns using the classifier trained by the method of FIG. 3;

FIG. 6 is a flow diagram of another example method for training a classifier for detection of slowing patterns in EEG data;

FIG. 7 is a flow diagram of a method for detection of slowing patterns using the classifier trained by the method of FIG. 6;

FIG. 8 is a flow diagram of a further example method for training a classifier for detection of slowing patterns in EEG data;

FIG. 9 is a flow diagram of a method for detection of slowing patterns using the classifier trained by the method of FIG. 8;

FIG. 10A shows an example EEG trace containing high-amplitude artifacts;

FIG. 10B shows an example EEG trace containing eye blink artifacts;

FIG. 11A shows another example EEG trace containing eye blink artifacts;

FIG. 11B shows an example EEG trace containing eye artifacts and Glossokinetic artifacts;

FIG. 12 shows an example EEG trace containing high amplitude noise from muscle and movement artifacts across multiple channels;

FIG. 13 is a scatter plot with four quadrants showing four different degrees of slowing;

FIG. 14 shows examples of EEGs with different degrees of slowing and a slow-free EEG (the percentage of slowing for each channel is displayed);

FIG. 15 shows boxplots of spectral characteristics of various EEG data sets used for training of classifiers in methods of the present disclosure; and

FIG. 16 shows an example user interface for slowing detection.

### DETAILED DESCRIPTION

Embodiments of the present disclosure relate to detection of slowing patterns in EEG data. Some embodiments relate to detection of EEG slowing in single-channel segments (channel-level detection), multi-channel segments (segment-level detection), or full EEGs (EEG-level detection). An EEG analysis system according to some embodiments can be deployed in a wide variety of contexts, for example for diagnosis of an underlying neurological condition, monitoring brain activity during anesthesia, or monitoring of patients in Intensive Care Units (ICUs).

An EEG analysis system according to some embodiments may take as input an entire EEG sample, and perform slowing classification to detect if an abnormal amount of slowing exists in the EEG sample. Additionally, it may detect clusters of slowing in EEG channels, or time stamps of segments in which slowing is present, to determine where and when the slowing occurs. This can allow clinicians and other expert users to review an EEG more easily, as the expert user can narrow down the abnormality and locate the anomaly more rapidly. Because of the time saved in EEG reviewing, more time is available for clinicians to tend to their patients.

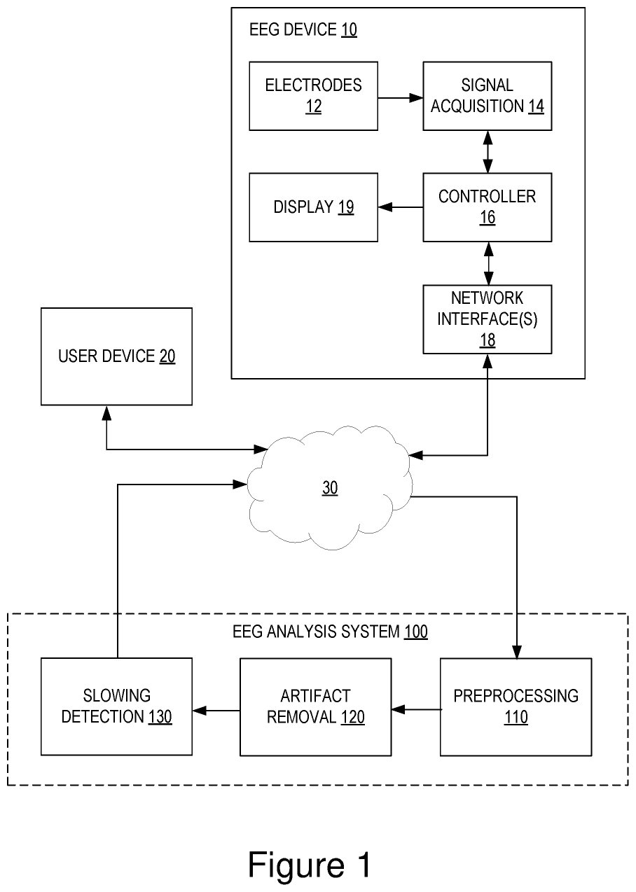

FIG. 1 shows an example block architecture of an EEG analysis system 100 according to some embodiments. The EEG analysis system 100 is in communication with an EEG device 10.

The EEG device 10 comprises electrodes 12 for attachment to a subject for acquisition, and in at least some cases processing, of electrical signals from the brain of the subject. This may be done by a signal acquisition module 14 to which the electrodes 12 are connected. The signal acquisition module may comprise an amplification component for amplifying the raw signals recorded by electrodes 12.

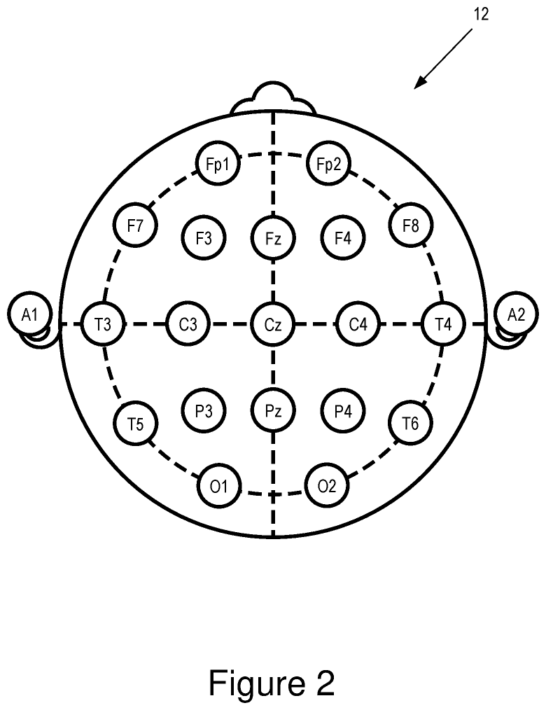

An example placement of electrodes 12 is shown in FIG. 2. This electrode placement corresponds to the standard international 10-20 system, the “10” and “20” referring to the fact that the actual distances between adjacent electrodes are either 10% or 20% of the total front-back or right-left distance of the skull. The layout in FIG. 2 has 21 electrode positions. It will be appreciated, however, that many other electrode placements are possible, and that fewer or more electrodes may be used. For example, higher resolution systems that use a 10% division or 5% division (filling in spaces between the positions shown in FIG. 2) are also used.

The EEG device 10 records brain waves from different amplifiers using various combinations of electrodes called montages. A montage is a particular arrangement of electrode connections, whereby pairs of electrodes are linked by connecting them to the inputs of respective amplifiers. The amplified difference in signals from the two electrodes constitutes a single channel of the EEG output. For example, in a bipolar montage, consecutive pairs of electrodes are linked by connecting the electrode input 2 of one channel to input 1 of the subsequent channel, so that adjacent channels have one electrode in common. The bipolar chains of electrodes may be connected going from front to back (longitudinal) or from left to right (transverse). In a bipolar montage signals between two active electrode sites are compared resulting in the difference in activity recorded. Another type of montage is the referential montage or monopolar montage. In a referential montage, various electrodes are connected to input 1 of each amplifier and a reference electrode is connected to input 2 of each amplifier. In a reference montage, signals are collected at an active electrode site and compared to a common reference electrode. One example of a referential montage is the common average reference (CAR) montage, which is used in embodiments of the present disclosure.

The number of electrodes determines the number of channels for an EEG. A greater number of channels produces a more detailed representation of a patient's brain activity. As noted above, each channel in the output from the EEG device 10 is the difference in electrical activity detected by two of the electrodes.

Returning to FIG. 1, the EEG device 10 further comprises a controller 16, which may comprise at least one processor, and may also comprise storage for storing signals acquired by signal acquisition module 14. The storage may also store instructions for controlling various components of the EEG device 10. For example, the controller 16 may be configured (via said instructions) to cause the signal acquisition module 14 to begin or end acquiring signals, and to do so at a desired sampling rate. The controller 16 may also be configured to send results of processing of the acquired signals to a display 19, and/or to external devices via one or more network interfaces 18. For example, a network interface 18 of the EEG device 10 may transmit data to a user device 20 via a network 30, which may be the public Internet. In this way, a user, such as a clinician, operating the user device 20 and who is remote from the EEG device 10, may still be able to observe EEG signals from the subject for remote monitoring purposes.

The EEG device 10 may be in the form of an EEG apparatus of the kind typically used in clinical practice, which comprise dedicated a control computer and amplifier unit, and require an expert user such as a nurse or doctor to place electrodes on the subject's head for recording. More recently, EEG headsets that are easier to use and that may be used for non-clinical purposes (such as gaming and marketing) have become available, and it is also contemplated that embodiments may be used in conjunction with any such headsets, or any other EEG systems that use surface electrodes.

As shown in FIG. 1, the EEG analysis system 100 is in communication with the EEG device 10 over a network 30. The network 30 may be a wide-area network such as the public Internet, or a local area network. In some embodiments, the EEG analysis system 100 may communicate directly with the EEG device 10, via a wireless (e.g. Bluetooth) or wired connection. In other embodiments, EEG recordings may be made by the EEG device 10, stored, and then analysed “offline” by the EEG analysis system 100.

In some embodiments, the EEG analysis system 100 may be in the form of one or more networked computing systems, each having a memory, at least one processor, and at least one computer-readable non-volatile storage medium (e.g., solid state drive), and the processes described herein may be implemented in the form of processor-executable instructions stored on the at least one computer-readable storage medium. However, it will be apparent to those skilled in the art that the processes described herein can alternatively be implemented, either in their entirety or in part, in one or more other forms such as configuration data of a field-programmable gate array (FPGA), and/or one or more dedicated hardware components such as application-specific integrated circuits (ASICs).

The one or more networked computing systems may receive EEG data from one or more EEG devices 10 via the network 30, analyse the EEG data, and transmit the results of the analysis back to the one or more EEG devices 10 and/or to one or more user devices 20. For example, an EEG device 10 may transmit all or part of an EEG to the EEG analysis system 100 for processing, and receive an analysis result in response.

In some embodiments, EEG data may be transmitted segment-by-segment by the EEG device 10 to the EEG analysis system 100, and each segment (and/or channels thereof) may be classified (as exhibiting slowing or not) in real-time. This may be advantageous where, for example, the EEG device 10 is being used to monitor an ICU patient, or a patient under anaesthesia during surgery.

In other embodiments, the EEG analysis system 100 may be integrated with the EEG device 10. For example, modules of the analysis system 100 may be implemented in the form of computer-readable instructions stored on storage of the controller 16 and configured to cause at least one processor of controller 16 to perform the processes described herein.

The EEG analysis system 100 comprises a preprocessing module 110 that receives raw EEG data from the EEG device 10, and performs various preprocessing steps such as downsampling, filtering, and montage configuration. It will be appreciated that some or all of such preprocessing may be performed by the EEG device 10 itself.

The EEG analysis system 100 also comprises an artifact removal module 120 that performs a sequence of artifact removal processes on the EEG sample to generate a preprocessed EEG sample. The sequence may comprise removal of one or more ocular artifacts and removal of one or more electrode artifacts.

The EEG analysis system 100 further comprises a slowing detection module 130 that analyses the preprocessed EEG sample to detect the presence of slowing. The detection may comprise extracting a first feature set from the preprocessed EEG sample; and passing the first feature set to a first trained classifier to predict whether the EEG sample contains abnormal slow waves or not. The prediction may be done on a channel-wise basis, for each segment of the EEG sample. As used herein, a “segment” is a portion of an EEG recording in one or more channels in a specific time window, such as in a 5 second window of the EEG recording. A segment of an EEG recording in a single channel may be referred to as a single-channel segment. Successive segments may partially overlap.

The first trained classifier may be a threshold-based classifier, a shallow learning model (such as a support vector machine or random forest-based classifier), or a deep learning model such as a convolutional neural network. Various implementations of such classifiers will be described in further detail below.

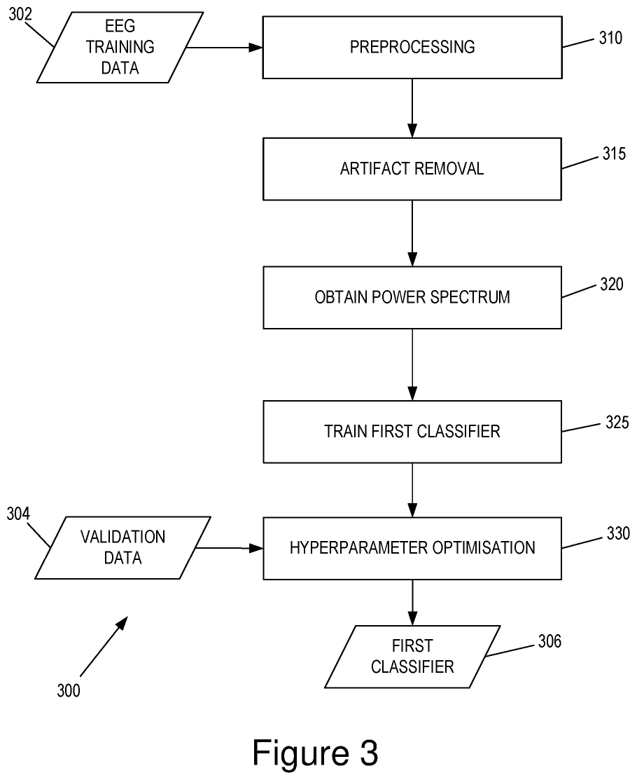

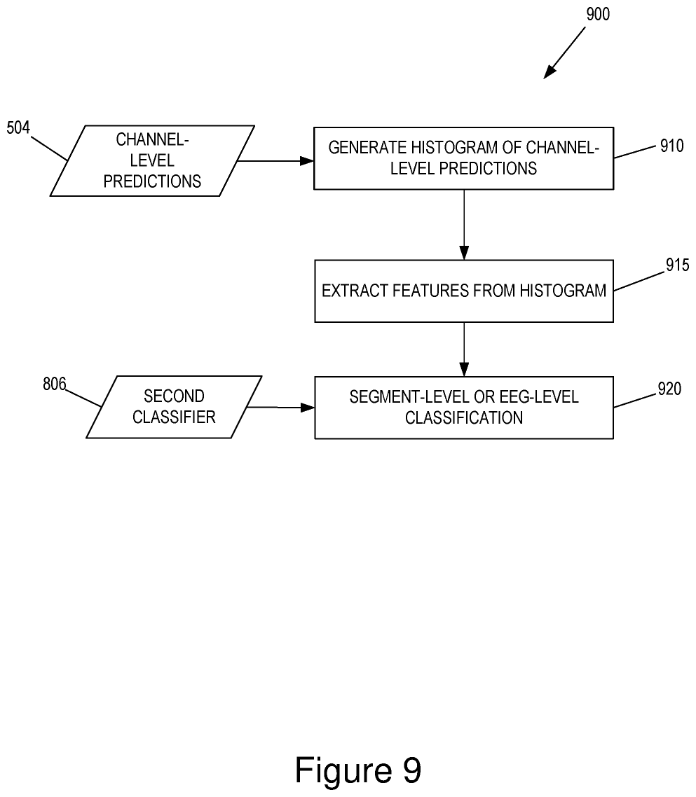

Various embodiments of a method for detecting the presence of slowing patterns in an EEG sample will now be described with reference to FIGS. 3 and 9.

FIG. 3 is a flow diagram of a process 300 for training a first classifier 306 using a training data set 302. The first classifier 306 is trained to provide a channel-wise classification of one or more segments of an EEG sample. The training data set 302 comprises labelled examples of EEG samples, where each sample is divided into segments, and each channel of each segment is associated with a binary indication as to whether it exhibits slowing or not. The segment is also annotated with an overall label as to whether it exhibits slowing or not. A smaller validation data set 304 is also used as part of the process 300, and is labelled in the same way as for the training data set 302. Examples of training data sets with which embodiments of the present disclosure have been used will be described in the experimental section below.

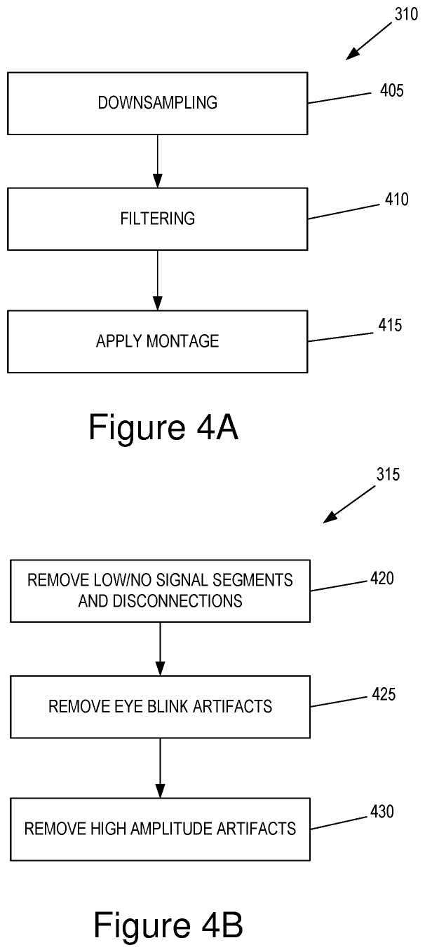

The process 300 begins at block 310 by preprocessing (e.g., by preprocessing module 110) the EEG samples of the training data set 302. Example preprocessing operations 310 are shown in FIG. 4A, and may comprise downsampling 405. Downsampling 405 may downsample the data to 128 Hz, for example, to reduce computational complexity. This may be followed by a filtering operation 410 to remove power-line interference, baseline drifts, high-frequency noise, and low-frequency noise. Finally, a montage, such as the CAR montage, may be applied at block 415. In the CAR montage, an average of all of the electrode signals is used as the reference. Other references, such as the Cz electrode, may be used to apply the montage.

The process 300 continues to block 315, where an artifact removal process is performed (e.g., by artifact removal module 120). The artifact removal process 315 comprises a sequence of artifact removal operations, as shown for example in FIG. 4B.

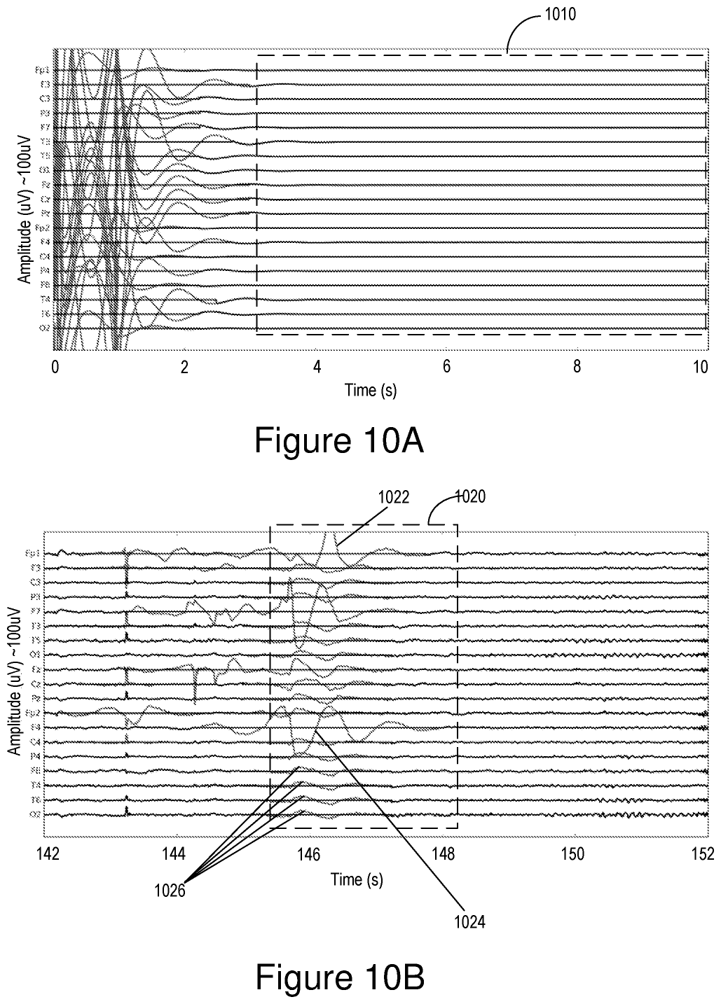

For example, the artifact removal operations may comprise removing low/no signal segments and disconnection segments 420. This artifact removal operation comprises identifying the EEG segments that contain no to low signal (under 0.001 uV) and removing them from the EEG. Segments that are disconnected are identified and removed from the EEG. Depending on the system, a disconnected segment may be identified as one having no signal, or one having constant voltage across all channels (with or without a slight variation). Every single-channel segment of the EEG is analysed to verify if the segment is disconnected or has useful EEG signals. An example of disconnection artifacts and no-signal segments is shown at 1010 in FIG. 10A.

The artifact removal operations may also comprise removing eye blink artifacts 425. To remove eye blink artifacts, single-channel segments are extracted for each channel using a sliding window of 500 milliseconds with 75% overlap. The same time window is used for each channel. One or more statistical properties of the single-channel segments extracted from electrode Fp1 and Fp2 are then checked, after smoothing the signal by applying a moving average box on the single-channel segments. The one or more statistical properties comprise at least the Pearson correlation, and may also comprise one or more of the range, gradient, and zero crossing. The statistical properties can be used to determine if the morphology of the waveform is indicative of an eye blink. If the two waveforms from Fp1 and Fp2 are highly correlated (for example, correlation>0.96), the single-channel segments for those channels can be deduced as those in which symmetrical eye blinks are present. Those single-channel segments are then removed from the EEG.

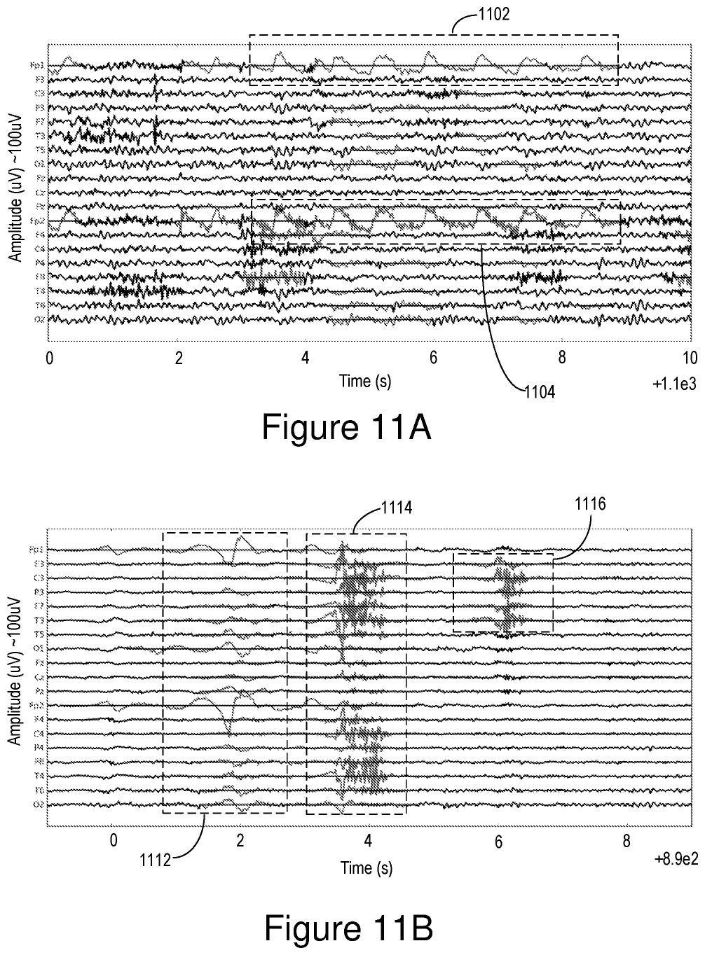

Additionally, if the blinks are forced or slow blinks, they can be much higher in amplitude and slower in frequency, allowing them to be detected by other neighboring electrodes (i.e., proximate to Fp1 and Fp2). Such leakage will appear as an attenuation blink waveform in other channels and can appear with the opposite polarity, depending on the electrode's location. Hence, the absolute Pearson correlation between the signals in Fp1 and Fp2 and the other single-channel segments can be determined after applying a moving average box. This enables a determination as to whether any spikes or slow waves in other channels, within the same time window, were induced by the two most frontal electrodes Fp1 and Fp2. FIG. 10B shows an example of eye blink artifacts rejected by an eye blink artifact removal process. The region 1020 encompasses high-amplitude eye blink artifacts for channel Fp1 (1022) and channel Fp2 (1024), with leakage to the other channels as shown, for example, at 1026. Eye blink artifacts may also be seen in the example of FIG. 11A, in dotted outline at 1102 for channel Fp1 and in dotted outline at 1104 for channel Fp2.

The artifact removal operations may further comprise removing high amplitude artifacts 430. This artifact removal operation identifies abnormally high amplitude artifacts and removes them from the EEG. Single-channel segments are extracted using a sliding window of 1 s with 75% overlap from each channel. For each single-channel segment, the root mean square (rms) amplitude is calculated before the channel-wise median and standard deviation (std) rms amplitude is computed. The channel rms amplitude threshold is calculated for each channel by adding a multiplier of the std to the median. The thresholds are calculated channel-wise as the rms amplitude of each channel is expected to be different, as signals from channels such as Fp1 and Fp2 (and even 01 and 02 if the subject has a smaller head) are less attenuated by the hair and will give a much greater rms amplitude by default.

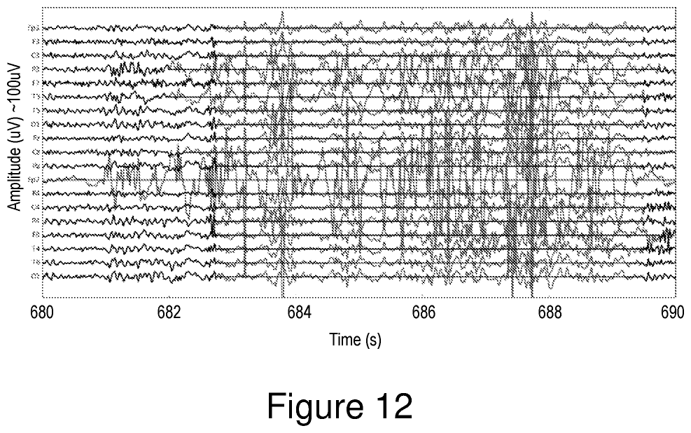

Some of the rejected artifacts from operation 430 are illustrated in FIG. 11B and FIG. 12. In FIG. 11B, an ocular artifact is detected in the region 1112, while Glossokinetic artifacts are seen at regions 1114 and 1116. FIG. 12 exhibits high amplitude noise from muscle and movement artifacts across multiple channels.

Returning to FIG. 3, once preprocessing 310 and artifact removal 315 have been performed, a power spectrum is obtained for each channel of each preprocessed EEG sample of the training data 302, at block 320. For each channel, the time-domain signal is transformed to a frequency domain signal having the range [0, 64] Hz, using methods known in the art (e.g., a Fast Fourier Transform). In some embodiments, the spectrum may be truncated, e.g. to the range [0, 30] Hz, to eliminate the gamma band component. In the context of slowing, the gamma band typically does not produce enough signal power to be a significant feature for slowing detection, and so it is removed to reduce the computational complexity of the process.

At block 325, a first classifier is trained, the first classifier being configured for channel-level detection of slowing.

The first classifier may be trained to classify a channel of a segment as containing slow waves or not, based on a first feature set. The first feature set may comprise one or more spectral features that are based on relative power values. For example, the features may be selected from the following relative power values and power ratio values:

TABLE 1

| Type | Spectral feature | Symbol/Equation |
|---|---|---|
| Relative | Delta RP | δ |
| Power | Theta RP | θ |
| (RP) | Alpha RP | α |
| | Beta RP | β |
| Power | Power Ratio Index (PRI) | (δ + θ)/(α + β) |
| Ratio | Delta Alpha Ratio (DAR) | δ/α |
| | Theta Alpha Ratio (TAR) | θ/α |
| | Theta Beta Alpha Ratio | θ/(α + β) |
| | (TBAR) | |

The frequency band definitions of the EEG are as follows: delta ([1,4]Hz), theta ([4,8]Hz), alpha ([8,13]Hz), and beta ([13,30]Hz). To determine the relative power, each band's power is computed, and the total power of the bandwidth ([1,30]Hz) is computed. Then, the relative power (RP) of each frequency band is calculated by dividing the frequency band's respective power by the total power.

In one example, the first classifier is a threshold-based classifier, and the first feature set contains a single feature that is selected from the spectral features above, such as PRI. A threshold-based classifier outputs a classification result based on comparing the selected spectral feature to a threshold value.

In some embodiments, the threshold-based classifier uses the distribution of spectral features across the EEG to perform classification. From the classification results, the threshold for classification can be selected. For example, the threshold can be selected based on an ROC curve generated from the classification results.

In some embodiments, the first classifier is a “shallow learning” model, such as a support vector machine (SVM), a logistic regression model, a random forest model, or a feedforward neural network having a single hidden layer. In these embodiments, the first feature set may comprise a plurality of spectral features, for example all of the eight spectral features mentioned above. The shallow learning model may be trained in any suitable fashion, for example by gradient descent or sub-gradient descent (for a SVM), maximising the likelihood (for logistic regression), bagging (for random forests), and so on.

In other embodiments, the first classifier is a “deep learning” model, such as a convolutional neural network (CNN). For example, a CNN may accept the entire power spectrum of the channel/segment at its input layer. In this case, the first feature set is not explicitly specified a priori, but is instead extracted automatically from the input layer.

In some embodiments, the CNN comprises 1D convolution filters with Rectified Linear Units (ReLU) as the activation functions. The outputs of these activation functions together form spectral feature maps. The dimensions of the feature maps are reduced by max-pooling. Next, the features are flattened and fed into a fully connected layer. The fully connected layer outputs are mapped into [0,1] with a softmax function.

In some embodiments, the CNN is trained by arranging the training samples in mini-batches, the size of each of which is equal to half the number of slowing waveforms in the training set 302. To prevent overfitting, balanced training can be applied by generating mini-batches with the same number of randomly selected slow waveforms and background waveforms. Additionally, a dropout of 0.5 can be applied in the fully connected layer. Training in each batch may be performed by gradient descent with backpropagation, for example. Cross-entropy may be used as the objective function for training the CNN. In some embodiments, the Adam optimiser may be used to optimise the learning rate.

Some embodiments may comprise optimising hyperparameters of the first classifier, at block 330. For example, the hyperparameters of a CNN may be optimised by applying a nested cross-validation (CV) on the training data. For example, 80% of the training data (i.e. training data set 302) may be utilised for learning the classifier parameters at block 325. The rest (i.e. validation data set 304) may be used for validation for selecting the CNN hyperparameters and for training termination criteria. To this end, various values of the hyperparameters may be used, and the CNN trained with the different values of the hyperparameters. The hyperparameters may be selected by finding the values that provide the best results on the validation data set 304. The CNN training is halted when the validation cost is minimised.

Some examples of hyperparameters of a CNN used with embodiments of the present disclosure are provided in Table 2 below.

TABLE 2

| Parameters | Values/Type |
|---|---|
| Number of convolution | 1, 2, 3 |
| layers | |
| Number of fully | 1, 2, 3 |
| connected layers | |
| Number of convolution | 8, 16, 32, 64, 128 |
| filters | |
| Dimension of | 1 × 3, 1 × 5, 1 × 7, 1 × 9, |
| convolution filters | 1 × 11 |
| Number of hidden | 16, 32, 64, 128, 256, |
| neurons | 512 |
| Activation functions | Softmax |
| Dropout probability | 0.4 |
| Maximum number of | 10000 |
| iterations | |
| Optimizer | Adam |
| Learning rate | 0.0001 |
| Measure | Cross-entropy |

Once training (and if performed, hyperparameter optimisation) is complete, parameters of a trained first classifier 306 are output.

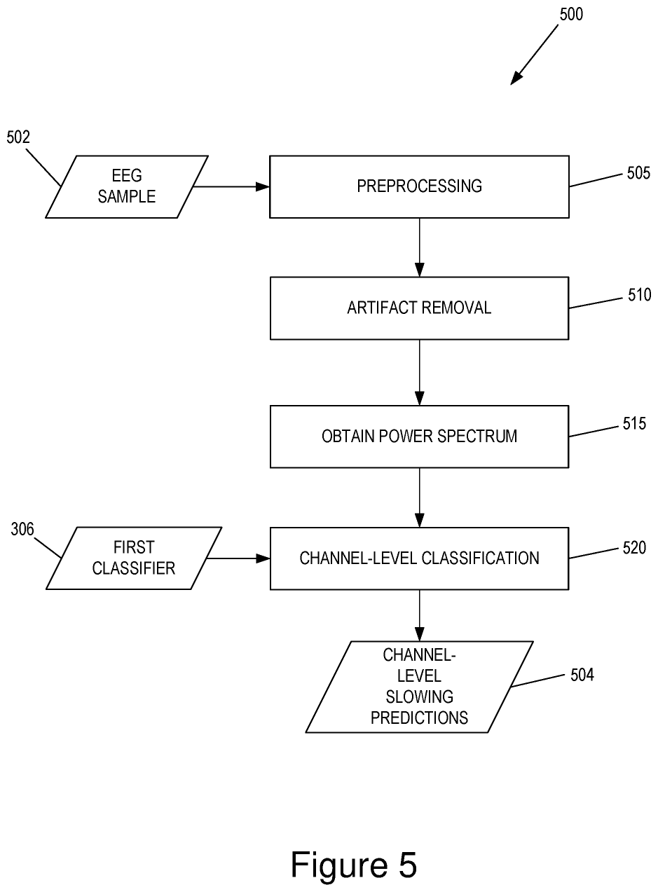

Turning now to FIG. 5, the use of a trained first classifier in a method 500 for channel-wise detection of slowing in an EEG sample will be described.

At block 505, the raw EEG sample is received and is preprocessed, in the same fashion as at block 310 in FIG. 3. The EEG sample may be a segment of an EEG recording, or may be an entire EEG recording.

At block 510, the preprocessed sample is subjected to artifact removal, in the same way as at block 315 in FIG. 3.

At block 515, the power spectrum of the preprocessed sample is obtained. If not done already, the preprocessed sample may be divided into segments prior to obtaining the power spectrum. A power spectrum may be obtained for each channel for each segment of the preprocessed EEG sample.

Next, at block 520, a channel-level classification is performed, using the parameters of a first classifier 306. The first classifier 306 may be trained according to the method 300 of FIG. 3, or by some other method. The first classifier 306 is applied to each channel of each segment, to obtain channel-level slowing predictions 504 for each segment.

For example, if the first classifier is a threshold-based classifier, then a selected spectral feature, such as the PRI, is determined for each channel for each segment, and is compared to the corresponding threshold of first classifier 306, to classify the segment as exhibiting slowing (or not). If the first classifier is a shallow learning model, for example a SVM, then a set of features (such as the eight spectral features listed in Table 1) is extracted from the power spectrum for each channel for each segment, and the parameters of the shallow learning model 306 are applied to the set of features to generate a channel-wise slowing prediction for each segment. If the first classifier is a deep learning model, such as a CNN, then the entire power spectrum for each channel, for each segment, is passed to the deep learning model 306 to generate a channel-wise slowing prediction for each segment.

In some embodiments, the degree of slowing along each EEG channel may be determined. This enables visualisation of the percentage of slowing in each EEG channel in the form of a scalp plot. This in turn allows a determination of the degree and location of slowing in the patient, which can be extremely useful in EEG reviewing and annotation processes.

The channel-level slowing detector (first classifier) 306 provides fine-grain information about slowing in the EEG, as it determines when and where slowing occurs in the EEG. This enables detection of different degrees of slowing, yielding more information for experts such as clinicians to assess the EEG slowing in a patient.

Four degrees of slowing can be distinguished from the EEG slowing duration (intermittent or continuous) and localization (focal or generalized). Following the literature, 20% can be set as the lower limit for abnormal slowing. Any channels that exhibit slowing for longer than 20% of the recording are marked as abnormal. If the number of abnormal channels is more than 50% of the total number of channels, the EEG exhibits generalised slowing, otherwise the slowing is considered focal. Next, the average percentage of slowing duration in those abnormal channels is computed. If the percentage is over 90%, it is classed as continuous slowing, otherwise it is intermittent if the slowing is above 20% and below 90% of the recording. Usually, EEG slowing can be considered generalised if it occurs at more than half of the electrodes. However, in some special cases, it might be viewed as focal even if most electrodes exhibit slowing. For example, a right-hemispheric slowing from an earlier surgery, and left temporal intermittent slow waves, would be considered two separate focal pathologies.

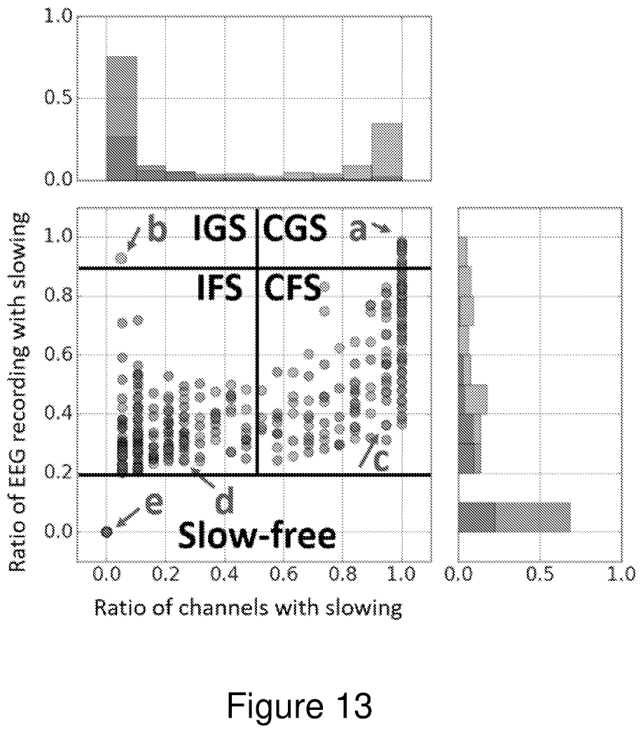

The four degrees of EEG slowing are illustrated in FIG. 13. The scatterplot is divided into four quadrants to reveal four regions: continuous generalized slowing (CGS), intermittent generalized slowing (IGS), continuous focal slowing (CFS), and intermittent focal slowing (IFS).

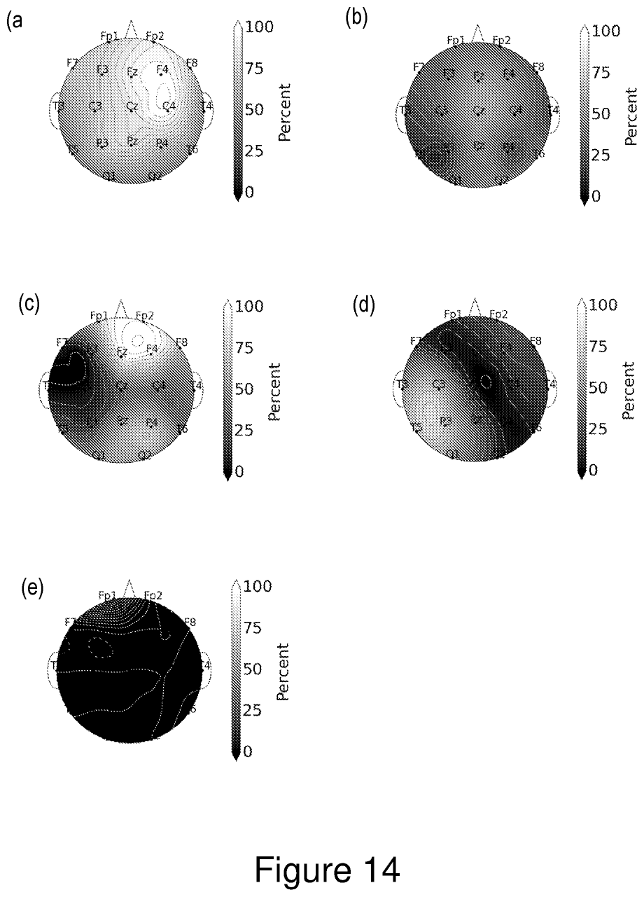

FIG. 14 illustrates five different examples of scalp heatmaps of the percentage of slowing, generated using classification results obtained using a deep learning model as the first classifier for channel-level prediction. FIG. 14(a) is an example of continuous and generalised slowing. FIG. 14(b) is an example of intermittent and generalised slowing. FIG. 14(c) is an example of continuous and focal slowing. FIG. 14(d) is an example of intermittent and focal slowing. FIG. 14(e) is an example of a slowing-free EEG.

With the four degrees of slowing defined, it is possible not only to perform slowing binary classification, but also to detect the degree of slowing in the EEG. This allows neurologists to apply the system of the presently disclosed embodiments for EEG reviewing process to allow faster annotation and to better understand the severity of the condition of the patient.

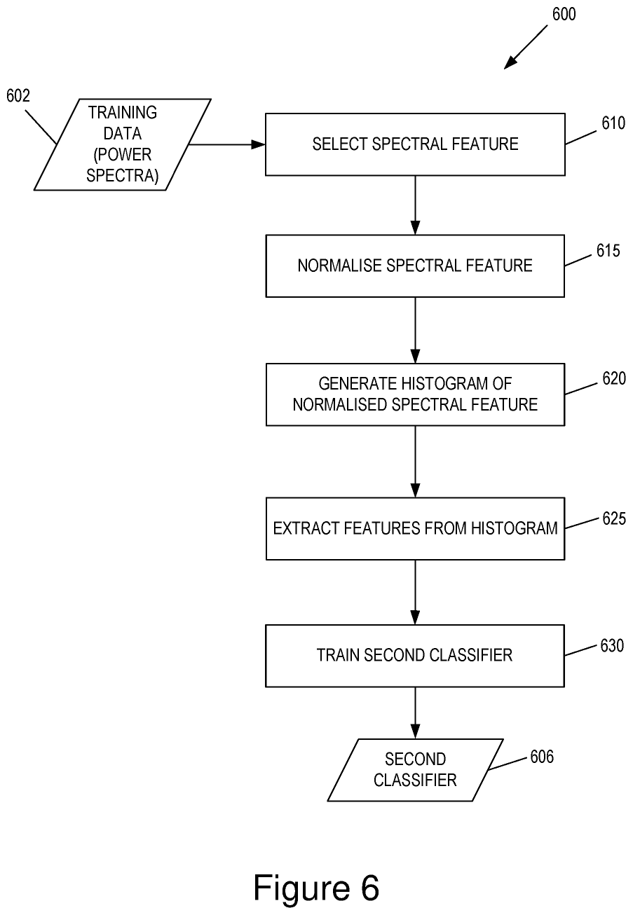

Turning now to FIGS. 6 to 9, various embodiments of methods for segment-level or EEG-level slowing detection will be described.

FIG. 6 shows an example of a method 600 for training of a segment-level or EEG-level classifier (second classifier) 606 for slowing detection.

The method 600 takes as input a training data set 602 comprising channel-wise power spectra of a segment, or of a plurality of segments, of a plurality of labelled EEG samples (where the segments are labelled as exhibiting slowing or not). For example, for EEG-level slowing detection, the EEG recording may be divided into a plurality of segments (e.g. of 5 seconds duration) with a 75% overlap. Further, a validation data set 604, that does not contain samples from the training set 602, may be used for optimising hyperparameters of the second classifier 606.

The method 600 may begin at block 610 by selecting a spectral feature to be used for classification. The spectral feature may be one of the relative power or power ratio features in Table 1, such as PRI. The value of this spectral feature is then determined for each channel. For example, for the 10-20 layout of FIG. 2 and using CAR montage, there will be 19 values for each segment, corresponding to the value of the spectral feature in the 19 channels.

Next, at block 615, as different spectral features have different ranges of values for slowing and slow-free EEGs, the spectral feature is normalised to ensure that most of the values for slow-free EEGs are bounded between approximately [0,1]. Normalisation may be performed by selecting one or more EEG recordings that are known not to contain slowing, finding the maximum values of respective spectral features in those slow-free EEGs, and dividing the respective spectral feature in the remainder of the data by the respective maximum value. The respective maximum values are also stored for use in subsequent normalisation of other samples.

Next, at block 620, a histogram is generated for the normalised spectral feature. To include the slowing portions exceeding the range of [0,1] (power ratio, PR, for slowing EEG is always greater than in slow-free EEG), the range is increased to [0,4]. Two further bins are added at [−100,0) and (4, 100] to include outliers. Each of the C*n values of the spectral feature (where C is the number of channels and n is the number of segments) are then placed in one of the bins covering the range [0,4], or in one of the outlier bins.

At block 625, one or more features of a second feature set are extracted from the histogram. The one or more features may comprise one or more of the mean, median, mode, standard deviation, minimum value, maximum value, range, kurtosis, and skewness of the histogram.

At block 630, the second classifier may be trained to classify a segment as containing slow waves or not, based on the second feature set. The second classifier may be a shallow learning model, such as a SVM, or a logistic regression model.

Once the second classifier is trained, its parameters 606 are output, and can be used to perform segment-level or EEG-level slowing detection on previously unseen samples.

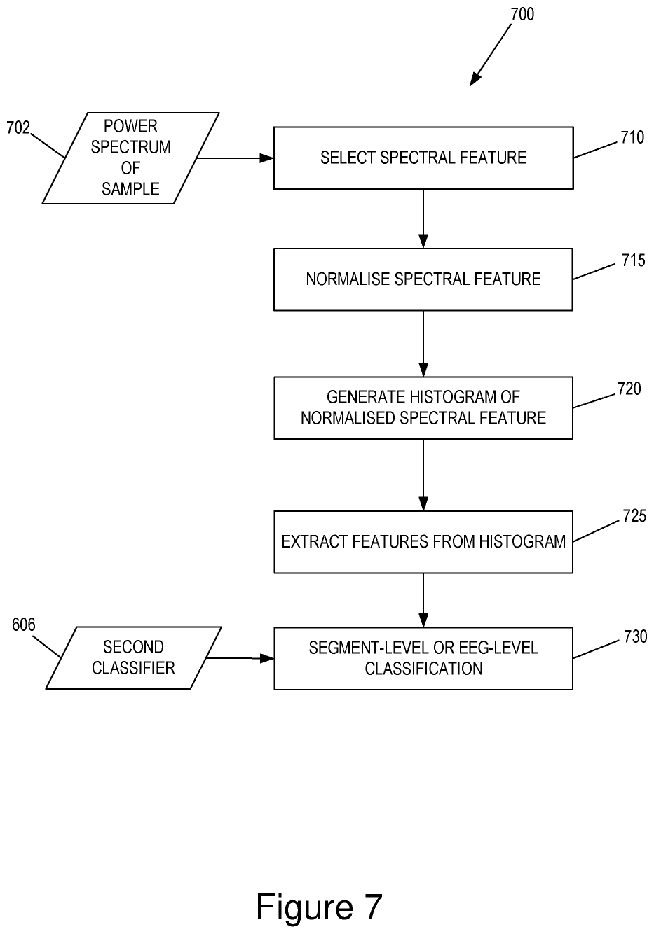

This can be seen in FIG. 7, in which channel-wise power spectra 702 of a sample on which segment-level or EEG-level slowing detection to be performed are input at block 710. A value of a selected spectral feature (such as PRI) is computed for each channel and for each segment in the sample 702.

At block 715, the values of the selected spectral feature have the same normalisation applied to them as was used for the training data 602, using the normalisation factor previously determined for the training data at block 615 of FIG. 6.

At block 720, a histogram is generated for the normalised spectral feature, in the same manner as done at block 620 of FIG. 6.

At block 725, a second feature set comprising the same features as were extracted for the histogram of the training data is obtained. The features of the second feature set are extracted for the histogram generated at block 720. The features may comprise one or more of the mean, median, mode, std, minimum value, maximum value, range, kurtosis, and skewness of the histogram.

At block 730, the parameters of the second classifier 606 are applied to the second feature set to generate the segment-level or EEG-level slowing classification.

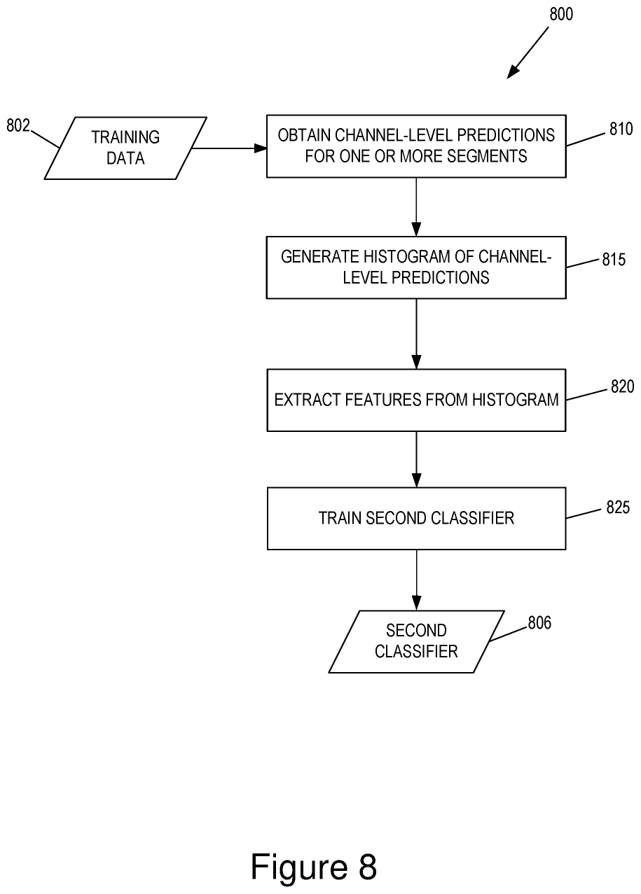

FIG. 8 shows another example of a method 800 for training of a segment-level or EEG-level classifier (second classifier) 806 for slowing detection. The method 800 may be used when channel-level predictions are available.

The method 800 may directly take as input, at block 810, a training data set 802 comprising the channel-level predictions (e.g. predictions 504) obtained by channel-level detection process 500 for one or more segments of EEG training data (e.g. the training data set 302). Alternatively, a training set of raw (labelled) EEG samples may be passed to the method 800 as training data set 802, and the channel-level detection process 500 may then be executed at block 810 to obtain the channel-level predictions.

Next, at block 815, a histogram of the channel-level predictions is generated.

At block 820, a second feature set comprising one or more features is extracted from the histogram. The features may comprise one or more of the mean, median, mode, std, minimum value, maximum value, range, kurtosis, and skewness of the histogram.

At block 825, the second classifier may be trained to classify a segment as containing slow waves or not, based on the second feature set. The second classifier may be a shallow learning model, such as a SVM, or a logistic regression model.

Once the second classifier is trained, its parameters 806 are output, and can be used to perform segment-level or EEG-level slowing detection on previously unseen samples.

Turning to FIG. 9, channel-level predictions for one or more segments of an EEG sample (for example, predictions 504 as obtained by the first classifier 306 applied in channel-level classification method 500) are input at block 910, in which a histogram of the channel-level predictions is generated, in the same way as for the histogram generated at block 815 of FIG. 8.

At block 915, a second feature set comprising the same features as were extracted for the histogram of the training data is obtained. The features may comprise one or more of the mean, median, mode, std (standard deviation), minimum value, maximum value, range, kurtosis, and skewness of the histogram.

At block 920, the parameters of the second classifier 806 are applied to the second feature set to generate the segment-level or EEG-level slowing prediction.

### Experimental Evaluation

In this study, 5 EEG dataset recordings from 5 different institutes from 3 different countries were analyzed. Most of the EEGs are between 20 to 40 minutes in duration.
1. TUH: Slow EEGs consist of majority persistent/frequent/continuous slowing and a mix of focal and generalized slowing. The EEGs are mostly from the ICU department. The TUH EEG Slowing Corpus (TUSL) is available at https://www.isip.piconepress.com/projects/tuh_eeg/html/downloads.shtml.
2. NNI: Slow EEGs consist of majority intermittent slowing and a mix of focal and generalized slowing. All the EEGs are from the outpatient department.
3. Fortis: Slow EEGs consist of majority intermittent slowing and a mix of focal and generalized slowing. All the EEGs are from the outpatient department.
4. LTMGH: The severity of slowing is not mentioned, and EEGs are only presenting generalized slowing. All the EEGs are from the outpatient department. The EEGs contains significantly more sweat artifact, causing a major difference in the frequency spectrum in the EEGs compared to those in the other datasets. Hence, this dataset will be evaluated separately.
5. NUH: Slowing is not mentioned in the clinical reports.

Characteristics of these data sets are shown in Table 3.

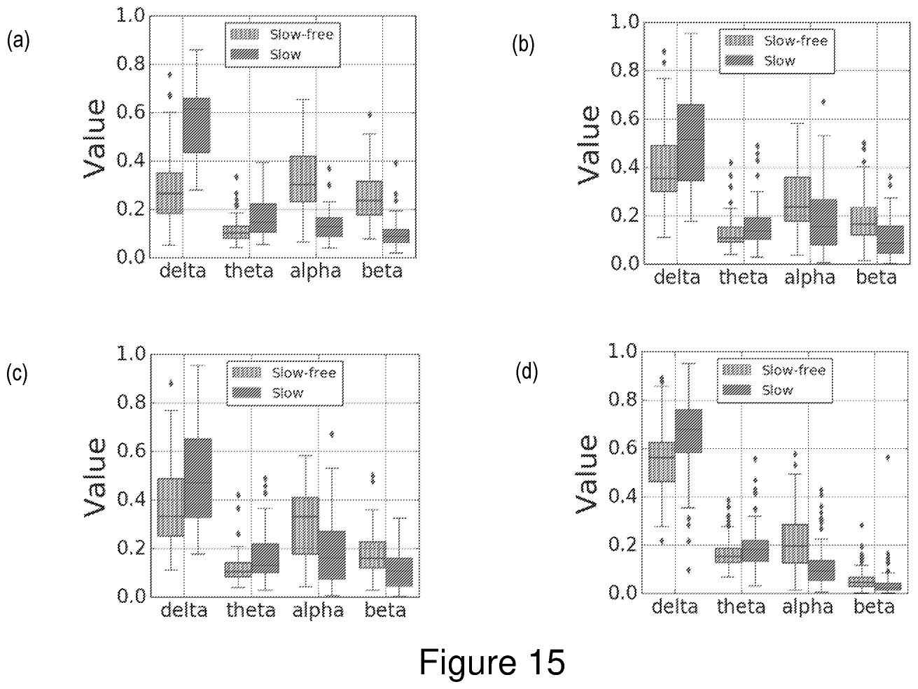

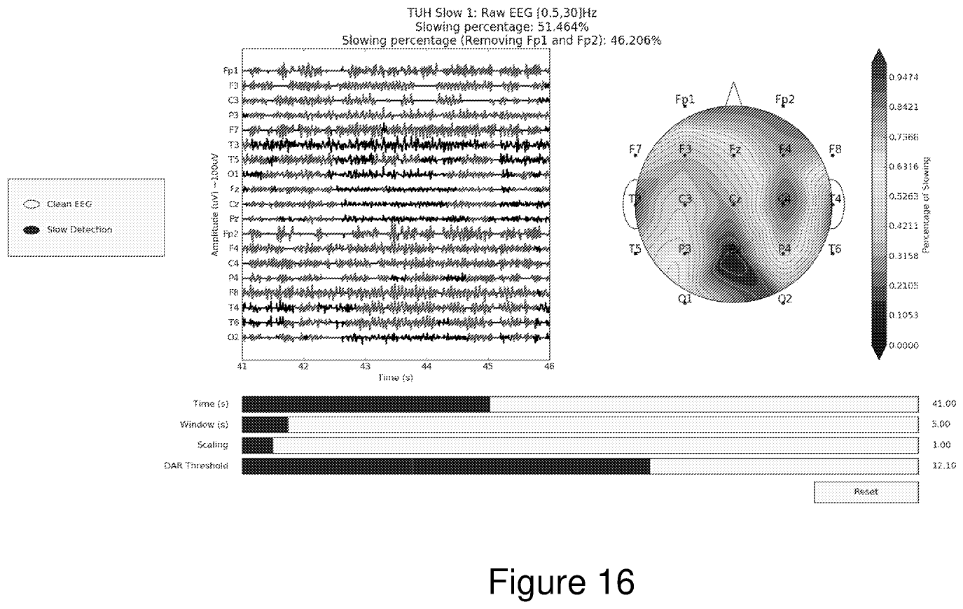

Comparing the difference in relative power between (a) TUH, (b) NNI, (c) Fortis, and (d) LTMGH datasets in FIG. 15, it was observed that the LTMGH data has a much higher delta power and much lower beta power. This can make the LTMGH EEGs more difficult to generalize across the other three datasets. Hence, the LTMGH dataset was considered separately.

TABLE 3

| | No. | Slow-free | | | | | Slowing | | | | |
|---|---|---|---|---|---|---|---|---|---|---|---|
| Dataset | of | | | | Duration | Age | | | | Duration | Age |
| Fs | EEG | Tot. | Gender | No | (minutes) | (years) | Tot. | Gender | No | (minutes) | (years) |
| TUH | 141 | 99 | M | 46 | 22.2 ± 4.4 | 42.0 ± 14.4 | 42 | M | 28 | 11.6 ± 6.1 | 53.0 ± 10.4 |
| 250, 256, | | | F | 53 | 21.3 ± 2.0 | 46.2 ± 16.9 | | F | 14 | 19.7 ± 4.1 | 47.5 ± 18.6 |
| 500 Hz | | | | | | | | | | | |
| NNI | 114 | 58 | M | 29 | 27.8 ± 0.6 | 45.6 ± 17.3 | 56 | M | 25 | 27.6 ± 1.6 | 51.2 ± 18.4 |
| 200 Hz | | | F | 29 | 27.4 ± 2.0 | 52.3 ± 19.9 | | F | 31 | 28.0 ± 1.3 | 53.0 ± 19.7 |
| Fortis | 935 | 655 | M | 155 | 20.9 ± 6.5 | 45.9 ± 19.7 | 280 | M | 50 | 20.3 ± 3.0 | 55.5 ± 17.8 |
| 500 Hz | | | F | 123 | 20.3 ± 4.1 | 45.7 ± 18.2 | | F | 19 | 20.6 ± 3.5 | 50.0 ± 17.0 |
| | | | UNK | 7 | 20.7 ± 1.0 | 43.0 ± 17.9 | | UNK | 4 | 22.2 ± 1.5 | 63.8 ± 5.3 |
| LTMGH | 1100 | 701 | M | 370 | 14.0 ± 1.5 | 33.5 ± 18.3 | 399 | M | 207 | 14.8 ± 1.9 | 37.0 ± 24.3 |
| 256 Hz | | | F | 331 | 14.3 ± 1.7 | 31.0 ± 18.7 | | F | 192 | 14.7 ± 2.6 | 36.8 ± 21.8 |
| Total | 1713 | 1143 | M | 600 | 17.1 ± 5.6 | 37.9 ± 19.2 | 570 | M | 310 | 16.4 ± 4.9 | 42.6 ± 23.3 |
| | | | F | 536 | 17.1 ± 4.6 | 37.1 ± 20.1 | | F | 256 | 17.0 ± 5.2 | 40.3 ± 22.0 |
| | | | UNK | 7 | 20.7 ± 1.0 | 43.0 ± 17.9 | | UNK | 4 | 22.2 ± 1.5 | 63.8 ± 5.3 |

| Dataset | | | | | Duration | Age |
|---|---|---|---|---|---|---|
| Fs | Total | Total | Gender | No | (minutes) | (year) |
| NUH | 150 | 150 | M | 89 | 19.4 ± 9.4 | 51.2 ± 19.9 |
| 250 Hz | | | F | 61 | 19.6 ± 9.3 | 56.5 ± 20.2 |

FIG. 15 shows boxplots of the average relative power (ARP) extracted. The boxplots indicate that the delta power is always higher in EEG with slowing as compared to without. Additionally, the delta and theta power are higher in EEG with slowing and hence have a lower alpha and beta power.

Leave-One-Subject-Out (LOSO) Cross-Validation (CV) and Leave-One-Institution-Out (LOIO) CV were used for validation. Two different CV schemes were performed for application reasons. For LOSO CV, access to some past EEGs (around 50 to 100 EEGs) and their clinical reports was assumed. With the data, the classification system can be retrained to perform predictions on EEGs from other patients from the same center in the future. To assess the system's performance in this scenario, LOSO CV was applied for each institute (dataset) separately by selecting one subject for testing and the remaining subjects for training the classification system. For LOIO CV, it was assumed that no EEGs nor clinical reports are available from a new center. Hence, existing datasets were used to train the classification system to predict those EEGs' labels from the new center. First, an institute of the pool of participating institutes (see above) was selected, and left out for testing. The EEGs from the remaining institutes were employed to train the classification system. This was repeated for each institution. To the best of the inventors' knowledge, this current study is the first to perform a cross-institutional assessment of automated EEG classification systems to detect pathological slowing. The LOIO CV assessment is important for evaluating the generalizability of the proposed system. Similarly, the LOSO CV is important for evaluation of the classification systems after recalibration for a particular dataset.

Embodiments of the classification system achieved EEG-level classification balanced accuracy of over 80.0% across four datasets via LOIO CV and over 80.0% across five datasets via LOSO CV.

Channel- and segment-level leave-one-LOSO and LOIO CV were performed on the channels and segments annotated in TUH, NNI, Fortis, and NUH datasets. Meanwhile, EEG-level LOSO and LOIO CV were performed on the EEGs from TUH, NNI, Fortis, and LTMGH datasets. The LTMGH dataset was not deployed during training on any scenario besides during LOSO CV on the dataset itself, as it may not generalize well across the other datasets.

The best results for the channel-, segment-, and EEG-level LOIO and LOSO CV for each system, together with their parameters, are displayed in Tables 3 to 5. The area under the receiver operating characteristic curve (AUC), balanced accuracy (BAC), sensitivity (SEN), and specificity (SPE) were used for evaluation. As the labels may be imbalanced, the results were evaluated mainly in terms of BAC.

$$Sensitivity = \frac{TP}{TP + FN}$$

$$Specificity = \frac{TN}{TN + FP}$$

$$Balanced Accuracy = \frac{1}{2} ( \frac{TP}{TP + FN} + \frac{TN}{TN + FP} ) = \frac{1}{2} ( Sensitivity + Specificity )$$

where TP, TN, FP, and FN are the true positive, true negative, false positive, and false negative, respectively.

In the following discussion, TDS refers to a threshold-based classifier, SLDS to a shallow-learning classifier, and DLDS to a deep-learning classifier.

In Tables 4 to 6, “CC” refers to channel-level classification, “Th PRI” refers to a threshold-based classifier that uses PRI as the classification feature, “LR” refers to logistic regression, “SVM_rbf” refers to a support vector machine using a radial basis function as its kernel, “RF” means random forests, “SC” refers to segment-level classification, “CNN” means a convolutional neural network, and Bins is the number of bins used for the segment-level or EEG-level classification. F is the number of filters and K is the kernel length (length of each filter window) for the CNN.

### Channel-Level Results

The DLDS performed the best for both LOIO and LOSO CV. The TDS that deploys thresholding on the PRI achieved the best LOIO and LOSO CV mean BAC, suggesting that PRI is the optimal feature for channel-level slowing identification.

TABLE 4

| | | LOIO Results | | | | | LOSO Results | | | | |
|---|---|---|---|---|---|---|---|---|---|---|---|
| System | Dataset | Parameters | AUC | AUPRC | ACC | BAC | Parameters | AUC | AUPRC | ACC | BAC |
| TDS | TUH | CC: Th PRI | 0.830 | 0.415 | 0.708 | 0.744 | CC: Th PRI | 0.830 | 0.415 | 0.837 | 0.763 |
| | NNI | | 0.819 | 0.698 | 0.748 | 0.733 | | 0.819 | 0.698 | 0.736 | 0.738 |
| | Fortis | | 0.633 | 0.250 | 0.652 | 0.585 | | 0.633 | 0.250 | 0.545 | 0.614 |
| | NUH | | 0.749 | 0.677 | 0.665 | 0.676 | | 0.749 | 0.677 | 0.690 | 0.685 |
| | Mean | | 0.758 | 0.510 | 0.693 | 0.684 | | 0.758 | 0.510 | 0.702 | 0.700 |
| SLDS | TUH | CC: LR | 0.862 | 0.575 | 0.752 | 0.772 | CC: SVM_rbf | 0.676 | 0.174 | 0.826 | 0.677 |
| | NNI | | 0.857 | 0.773 | 0.782 | 0.762 | | 0.828 | 0.695 | 0.773 | 0.776 |
| | Fortis | | 0.689 | 0.309 | 0.677 | 0.632 | | 0.626 | 0.308 | 0.648 | 0.602 |
| | NUH | | 0.786 | 0.782 | 0.712 | 0.709 | | 0.759 | 0.737 | 0.707 | 0.707 |
| | Mean | | 0.798 | 0.610 | 0.731 | 0.719 | | 0.722 | 0.479 | 0.739 | 0.691 |
| DLDS | TUH | CC: CNN | 0.827 | 0.349 | 0.655 | 0.723 | CC: CNN | 0.791 | 0.237 | 0.715 | 0.762 |
| | NNI | (F: 64, K: 13) | 0.847 | 0.732 | 0.768 | 0.768 | (F: 32, K: 7) | 0.837 | 0.667 | 0.738 | 0.765 |
| | Fortis | | 0.743 | 0.395 | 0.663 | 0.668 | SC: LR | 0.725 | 0.390 | 0.621 | 0.655 |
| | NUH | | 0.791 | 0.762 | 0.720 | 0.717 | Bins: 2 | 0.804 | 0.812 | 0.718 | 0.715 |
| | Mean | | 0.802 | 0.560 | 0.701 | 0.719 | | 0.789 | 0.527 | 0.698 | 0.724 |

### Segment-Level Results

The segment-level results are shown in Table 5.

For both LOIO and LOSO CV, the DLDS achieves the best mean BAC. The TDS and SLDS systems perform poorer than the DLDS. Similarly, employing PRI to construct the histograms yielded the best LOIO and LOSO CV results for the TDS.

### EEG-Level Results

The results for classification both with and without the LTMGH dataset are shown in Table 6.

Generally, the DLDS achieved the best mean BAC across all datasets, except for the LTMGH dataset. The TDS performed the best on the LTMGH dataset. The three systems achieved poorer results on the LTMGH dataset due to the other datasets spectral mismatch. Therefore, for EEGs with a frequency spectrum that deviate from the typical EEG spectrum characteristics, the EEG-level classification systems may be recalibrated for best results (LOSO CV).

TABLE 5

| | | LOIO Results | | | | | LOSO Results | | | | |
|---|---|---|---|---|---|---|---|---|---|---|---|
| System | Dataset | Parameters | AUC | AUPRC | ACC | BAC | Parameters | AUC | AUPRC | ACC | BAC |
| TDS | TUH | Feat: PRI | 0.761 | 0.517 | 0.732 | 0.678 | Feat: PRI | 0.827 | 0.690 | 0.809 | 0.769 |
| | NNI | SC: LR | 0.884 | 0.852 | 0.818 | 0.807 | SC: LR | 0.858 | 0.818 | 0.775 | 0.758 |
| | Fortis | Bins: 5 | 0.649 | 0.376 | 0.654 | 0.590 | Bins: 5 | 0.689 | 0.539 | 0.692 | 0.669 |
| | NUH | | 0.758 | 0.818 | 0.691 | 0.677 | | 0.692 | 0.749 | 0.644 | 0.658 |
| | Mean | | 0.763 | 0.641 | 0.724 | 0.688 | | 0.766 | 0.699 | 0.730 | 0.713 |
| SLDS | TUH | CC: LR | 0.812 | 0.598 | 0.784 | 0.753 | CC: SVM_rbf | 0.745 | 0.491 | 0.742 | 0.710 |
| | NNI | SC: LR | 0.896 | 0.868 | 0.831 | 0.821 | SC: RF | 0.845 | 0.732 | 0.822 | 0.818 |
| | Fortis | Bins: 2 | 0.694 | 0.428 | 0.692 | 0.664 | Bins: 5 | 0.586 | 0.351 | 0.650 | 0.582 |
| | NUH | | 0.77 | 0.81 | 0.699 | 0.69 | | 0.703 | 0.760 | 0.661 | 0.671 |
| | Mean | | 0.793 | 0.676 | 0.751 | 0.732 | | 0.720 | 0.584 | 0.719 | 0.695 |
| DLDS | TUH | CC: CNN | 0.767 | 0.466 | 0.745 | 0.758 | CC: CNN | 0.749 | 0.511 | 0.825 | 0.780 |
| | NNI | (F: 64, K: 13) | 0.842 | 0.771 | 0.817 | 0.811 | (F: 32, K: 7) | 0.851 | 0.772 | 0.829 | 0.832 |
| | Fortis | SC: LR | 0.765 | 0.547 | 0.754 | 0.742 | SC: LR | 0.747 | 0.455 | 0.723 | 0.715 |
| | NUH | Bins: 10 | 0.783 | 0.785 | 0.725 | 0.708 | Bins: 2 | 0.748 | 0.745 | 0.742 | 0.737 |
| | Mean | | 0.789 | 0.642 | 0.76 | 0.755 | | 0.774 | 0.621 | 0.780 | 0.766 |

The results in Table 6 illustrate that if there is no access to EEG reports for recalibration, the LOIO CV results suggest that the systems could evaluate the EEGs as reliably as a recalibrated system. Omitting the LTMGH dataset, the three systems achieved an LOIO CV mean BAC close to the LOSO CV mean BAC of 82.0% achieved by all three systems; the best BAC obtained given datasets. The DLDS achieves an almost identical mean BAC of approximately 82.0% for both LOIO and LOSO CV (excluding the LTMGH dataset). This implies that the DLDS can potentially perform equally well in both scenarios.

TABLE 6

| | | LOIO Results | | | | | LOSO Results | | | | |
|---|---|---|---|---|---|---|---|---|---|---|---|
| System | Dataset | Parameters | AUC | AUPRC | ACC | BAC | Parameters | AUC | AUPRC | ACC | BAC |
| TDS | TUH | Feat: PRI | 0.95 | 0.926 | 0.923 | 0.897 | Feat: 4 RP | 0.942 | 0.906 | 0.923 | 0.911 |
| | NNI | SC: GB | 0.71 | 0.786 | 0.728 | 0.724 | SC: GB | 0.76 | 0.775 | 0.746 | 0.744 |
| | Fortis | Bins: 20 | 0.847 | 0.677 | 0.863 | 0.796 | Bins: 20 | 0.846 | 0.706 | 0.872 | 0.806 |
| | LTMGH | | 0.714 | 0.637 | 0.698 | 0.611 | | 0.829 | 0.72 | 0.762 | 0.758 |
| | Mean | | 0.805 | 0.757 | 0.803 | 0.757 | | 0.844 | 0.777 | 0.826 | 0.805 |
| | Mean* | | 0.836 | 0.796 | 0.838 | 0.806 | | 0.849 | 0.796 | 0.847 | 0.820 |
| SLDS | TUH | CC: SVM | 0.946 | 0.895 | 0.923 | 0.911 | CC: RF | 0.919 | 0.897 | 0.895 | 0.884 |
| | NNI | SC: LR | 0.754 | 0.763 | 0.702 | 0.700 | SC: LR | 0.828 | 0.844 | 0.772 | 0.771 |
| | Fortis | Bins: 2 | 0.838 | 0.664 | 0.790 | 0.781 | Bins: 5 | 0.831 | 0.641 | 0.863 | 0.804 |
| | LTMGH | | 0.713 | 0.570 | 0.423 | 0.539 | | 0.743 | 0.609 | 0.732 | 0.716 |
| | Mean | | 0.813 | 0.723 | 0.710 | 0.733 | | 0.830 | 0.748 | 0.815 | 0.794 |
| | Mean* | | 0.846 | 0.774 | 0.805 | 0.797 | | 0.859 | 0.794 | 0.843 | 0.820 |
| DLDS | TUH | CC: CNN | 0.961 | 0.919 | 0.901 | 0.916 | CC: CNN | 0.943 | 0.853 | 0.922 | 0.917 |
| | NNI | (F: 64, K: 9) | 0.728 | 0.778 | 0.728 | 0.726 | (F: 32, K: 5) | 0.774 | 0.801 | 0.754 | 0.751 |
| | Fortis | SC: LR | 0.847 | 0.674 | 0.855 | 0.817 | SC: LR | 0.836 | 0.652 | 0.841 | 0.786 |
| | LTMGH | Bins: 10 | 0.598 | 0.390 | 0.376 | 0.506 | Bins: 15 | 0.723 | 0.573 | 0.704 | 0.690 |
| | Mean | | 0.783 | 0.690 | 0.715 | 0.741 | | 0.819 | 0.720 | 0.805 | 0.786 |
| | Mean* | | 0.845 | 0.790 | 0.828 | 0.820 | | 0.851 | 0.769 | 0.839 | 0.818 |

Many modifications will be apparent to those skilled in the art without departing from the scope of the present invention.

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A method for detecting presence of slowing patterns in an EEG sample comprising a plurality of channels of EEG signals, each channel comprising one or more segments, the method comprising:
obtaining a first classifier that is trained to classify EEG samples as containing abnormal slow waves or not;
performing a sequence of artifact removal processes on the EEG sample to generate a preprocessed EEG sample;
extracting a first feature set from the preprocessed EEG sample; and
passing the first feature set to the first classifier to predict whether the EEG sample contains abnormal slow waves or not;
wherein the sequence of artifact removal processes comprises removal of one or more ocular artifacts and removal of one or more electrode artifacts.

2. The method of claim 1, wherein removal of one or more electrode artifacts comprises: identifying and removing low signal segments; identifying and removing disconnected segments; and/or identifying and removing abnormal high-amplitude segments.

3. The method of claim 1, wherein removal of one or more ocular artifacts comprises removal of eye blink artifacts.

4. The method of claim 3, wherein removal of eye blink artifacts comprises determining a correlation between an Fp1 channel of the plurality of channels and an Fp2 channel of the plurality of channels in the preprocessed EEG sample in respective segments of said one or more segments; and removing, from the preprocessed EEG sample, any segments for which the correlation exceeds a threshold.

5. The method of claim 1, wherein the first classifier is applied separately to each of the plurality of channels to obtain a plurality of channel-wise slowing predictions.

6. The method of claim 5, comprising obtaining a second classifier that is trained to classify the one or more segments as containing abnormal slow waves based on a second feature set that is extracted from the first feature set, or from the plurality of channel-wise slowing predictions, or from both the first feature and from the plurality of channel-wise slowing predictions; and passing the second feature set to the second classifier to obtain a slowing prediction for the one or more segments or for the EEG sample as a whole.

7. The method of claim 1, wherein the first feature set comprises one or more spectral features, wherein each spectral feature is based on at least one relative power value that is a ratio of a power in a frequency band to a total power in one of the plurality of channels.

8. The method of claim 7, wherein the one or more spectral features comprise one or more of a set of power ratios comprising: power ratio index, PRI=(δ+θ)/(α+β); delta alpha ratio, DAR=δ/α; theta alpha ratio, TAR=θ/α; and theta beta ratio, TBAR=θ/(α+β); where α is relative power in the α frequency band, β is relative power in the β frequency band, δ is relative power in the δ frequency band, and θ is relative power in the θ frequency band.

9. The method of claim 6, wherein the second feature set comprises one or more statistical properties of the plurality of channel-wise slowing predictions.

10. The method of claim 7, wherein the second feature set comprises one or more statistical properties of the at least one relative power value, the power ratio, or both.

11. The method of claim 9, wherein the one or more statistical properties comprise one or more of: a histogram; a mean; a standard deviation; a minimum; a maximum; a range; a standard deviation of a gradient; and a standard deviation of a curvature.

12. The method of claim 1, wherein the first classifier is a support vector machine, a binary classifier based on thresholding, or logistic regression.

13. The method of claim 1, wherein the first classifier is a convolutional neural network (CNN).

14. The method of claim 6, wherein the second classifier is a support vector machine, logistic regression, or random forests.

15. The method of claim 5, comprising determining a percentage of slowing for each channel based on the plurality of channel-wise slowing predictions.

16. The method of claim 15, comprising generating a scalp heatmap of the percentage of slowing.

17. A system for detecting presence of slowing patterns in EEG data, the system comprising:
memory; and
at least one processor in communication with the memory;
wherein the memory has stored thereon computer-readable instructions for causing the at least one processor to perform a method comprising:
obtaining a first classifier that is trained to classify EEG samples as containing abnormal slow waves or not;
performing a sequence of artifact removal processes on the EEG sample to generate a preprocessed EEG sample;
extracting a first feature set from the preprocessed EEG sample; and
passing the first feature set to the first classifier to predict whether the EEG sample contains abnormal slow waves or not;
wherein the sequence of artifact removal processes comprises removal of one or more ocular artifacts and removal of one or more electrode artifacts.

18. A non-transitory computer-readable storage having stored thereon instructions for causing at least one processor to perform a method comprising:
obtaining a first classifier that is trained to classify EEG samples as containing abnormal slow waves or not;
performing a sequence of artifact removal processes on the EEG sample to generate a preprocessed EEG sample;
extracting a first feature set from the preprocessed EEG sample; and
passing the first feature set to the first classifier to predict whether the EEG sample contains abnormal slow waves or not;
wherein the sequence of artifact removal processes comprises removal of one or more ocular artifacts and removal of one or more electrode artifacts.
